(19) United States
(12) Patent Application Publication
(10) Pub. No.: US 2018/0289548 A1
(43) Pub. Date: Oct. 11, 2018

(54) Goggles with Ventilation Structure

(71) Applicant: Arthur Wang, Taipei City (TW)

(72) Inventor: Arthur Wang, Taipei City (TW)

(21) Appl. No.: 15/480,442

(22) Filed: Apr. 6, 2017

(51) Int. Cl. A61F 9/02

(52) U.S. Cl. CPC A61F 9/028; A61F 9/027; A61F 9/026

(57) ABSTRACT

Goggles with a ventilation structure have a frame, a lens, and a strap. The frame has multiple ventilation portions formed on an inner peripheral surface of the frame. The lens is mounted on the frame and has multiple through holes disposed along a peripheral edge of the lens and respectively communicating with the ventilation portions. The strap has two ends respectively connected to two opposite sides of the frame. Air outside the goggles can ventilate a space defined between the lens and a face of a wearer via the ventilation portions and the through holes, so as to dissipate heat accumulated in the space and to prevent the lens from fogging. Formation of the ventilation portions and the through holes does not require expanding a shape of the frame of the goggles. Thus, the goggles have compact volume and light weight and are comfortable for wearing.

### BACKGROUND OF THE INVENTION

### 1. Field of the Invention

The present invention relates to goggles, especially to goggles with a ventilation structure.

### 2. Description of the Prior Art(s)

Eyes are weaker parts of a human body. Goggles, such as snow goggles, wind goggles, sand goggles, and the like, are worn to enclose areas surrounding the eyes of a wearer, so as to prevent particulates, fluids, or chemicals from damaging the eyes. However, when a wearer wears the goggles, temperature differences between an inner side and an outer side of a lens of the goggles causes the inner side of the lens to become foggy easily and blurs vision of the wearer. In order to prevent accidents from happening, many anti-fogging designs are provided for the goggles.

With reference to FIGS. 6 and 7, conventional goggles include a frame 61, a ventilation sponge 62, a lens 63, a cushion pad 64, and a strap 65.

The frame 61 has an upper frame portion 611, a lower frame portion 612, a receiving recess, an inner bottom, multiple upper ventilation holes 613, and multiple lower ventilation holes 614. The receiving recess is formed in an upper surface of the upper frame portion 611. The inner bottom is defined in the receiving recess. The upper ventilation holes 613 are separately formed through the inner bottom of the upper frame portion 611. The lower ventilation holes 614 are separately formed through the lower frame portion 612.

The ventilation sponge 62 is embedded in the receiving recess and covers the upper ventilation holes 613. The lens 63 is attached to and covers a front of the frame 61 and has multiple front ventilation holes 631. The front ventilation holes 631 are formed through the lens 63 and are separately arranged along an upper edge of the lens 63. Each of the front ventilation holes 631 is covered with a ventilation sponge 632. The cushion pad 64 is mounted on a rear of the frame 61 and is disposed along a periphery of the frame 61. The strap 65 has two ends respectively connected to two opposite side portions of the frame 61.

With the upper ventilation holes 613, the lower ventilation holes 614, and the front ventilation holes 631, air outside an inner space defined between the lens 63 and a wearer's face can flow into the inner space via the lower ventilation holes 614. Then the air, together with heat accumulated in the inner space, flows out of the inner space via the upper ventilation holes 613 and the front ventilation holes 631, so as to prevent the lens 63 from fogging and to provide the wearer with clear vision.

However, in order to ensure that the conventional goggles have a wide vision area, the upper ventilation holes 613 on the frame 61, the front ventilation holes 631 on the lens 63, and the ventilation sponges 632 that cover the front ventilation holes 631 should be disposed outside the vision area BB formed on the conventional goggles. As shown in FIG. 6, the vision area BB is surrounded by a dashed line. Consequently, the conventional goggles are bulky and heavy.

To overcome the shortcomings, the present invention provides goggles with a ventilation structure to mitigate or obviate the aforementioned problems.

### SUMMARY OF THE INVENTION

The main objective of the present invention is to provide goggles with a ventilation structure. The goggles with a ventilation structure have a frame, a lens, and a strap. The frame has an inner peripheral recess and multiple ventilation portions. The inner peripheral recess is formed in an inner peripheral surface of the frame to divide the frame into a front frame strip and a rear frame strip. The ventilation portions are separately formed on the inner peripheral surface of the frame. Each of the ventilation portions has a front ventilation hole formed through the front frame strip of the frame and a rear ventilation hole formed through the rear frame strip of the frame. The lens is mounted on the frame and has multiple through holes disposed along a peripheral edge of the lens and respectively communicating with the ventilation portions. The strap has two ends respectively connected to two opposite sides of the frame.

Air outside the goggles can ventilate a space defined between the lens and a face of a wearer via the ventilation portions on the frame and the through holes on the lens, so as to dissipate heat accumulated in the space and to prevent the lens from fogging. Formation of the ventilation portions and the through holes does not require expanding a shape of the frame of the goggles . Thus, the goggles have compact volume and light weight and are comfortable for wearing.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of goggles with a ventilation structure in accordance with the present invention;

FIG. 2 is an exploded perspective view of the goggles in FIG. 1;

FIG. 3 is an enlarged upper cross-sectional view of a lens and an upper frame portion of a frame of the goggles in FIG. 1;

FIG. 4 is an enlarged cross-sectional side view of the goggles along line A-A in FIG. 3;

FIG. 5 is an enlarged lower cross-sectional view of a lens, a cushion pad, and a lower frame portion of the frame of the goggles in FIG. 1;

FIG. 6 is a perspective view of conventional goggles in accordance with the prior art; and

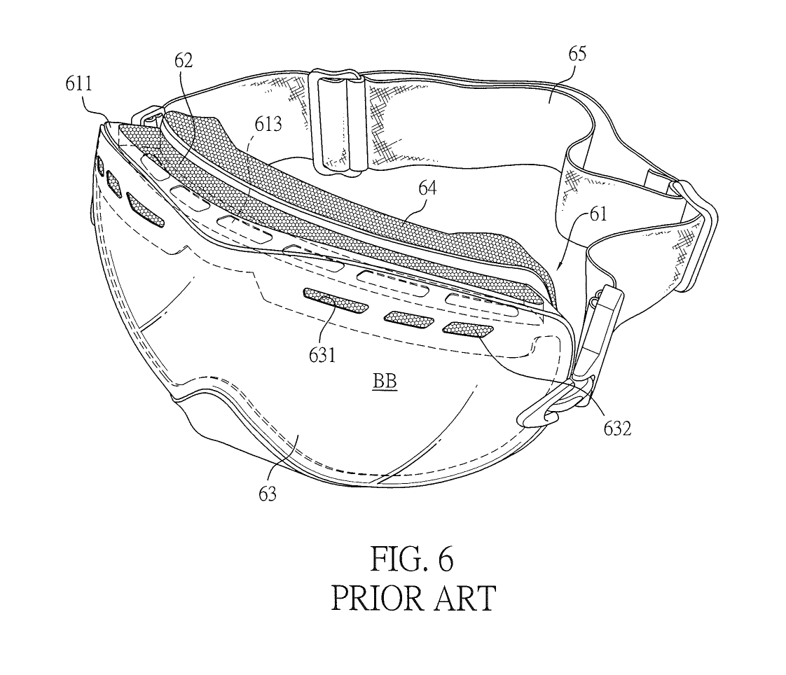

FIG. 7 is another perspective view of the conventional goggles in FIG. 6.

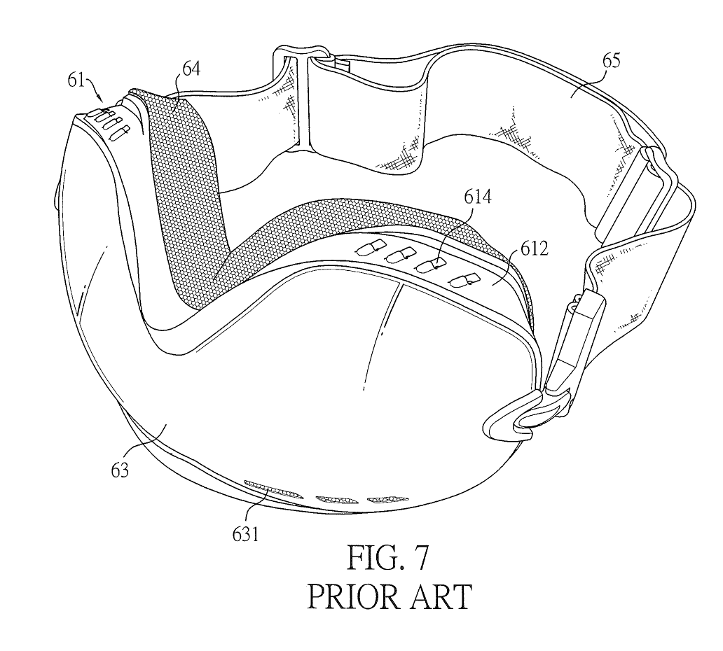

### DETAILED DESCRIPTION OF THE PREFERRED EMBODIMENTS

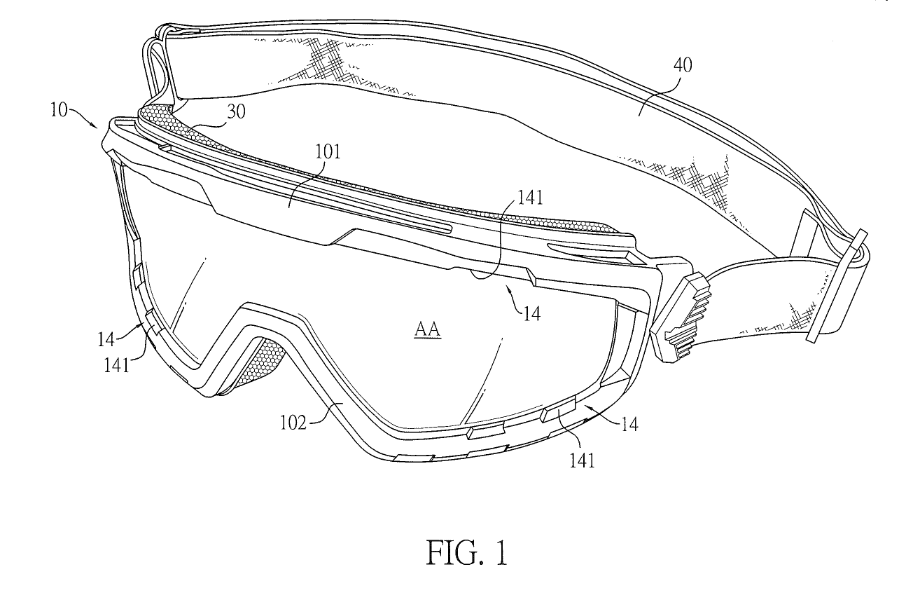

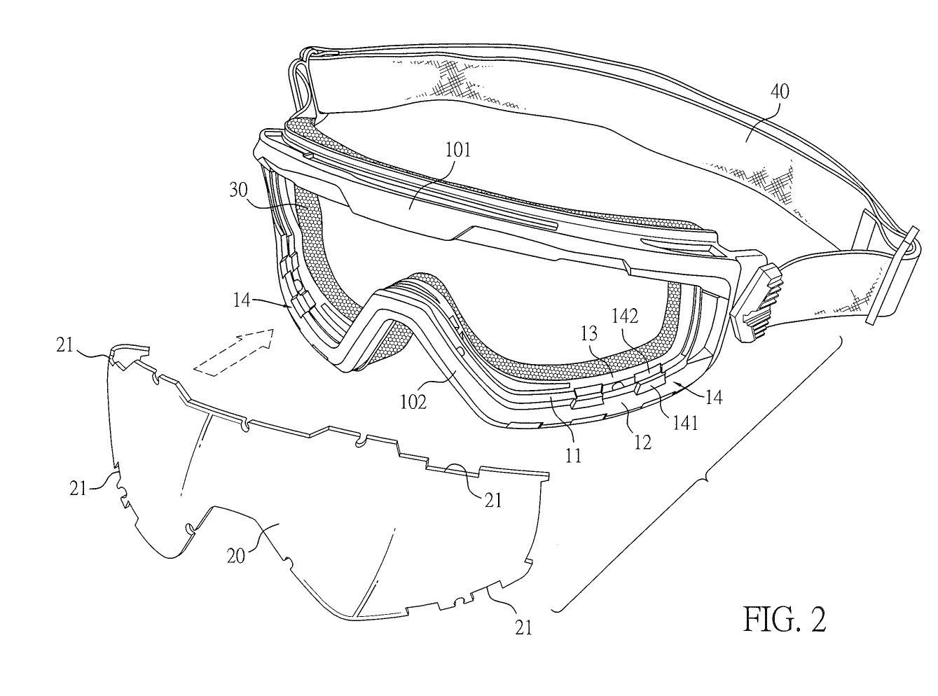

With reference to FIGS. 1 and 2, goggles with a ventilation structure in accordance with the present invention comprise a frame 10, a lens 20, a cushion pad 30, and a strap 40.

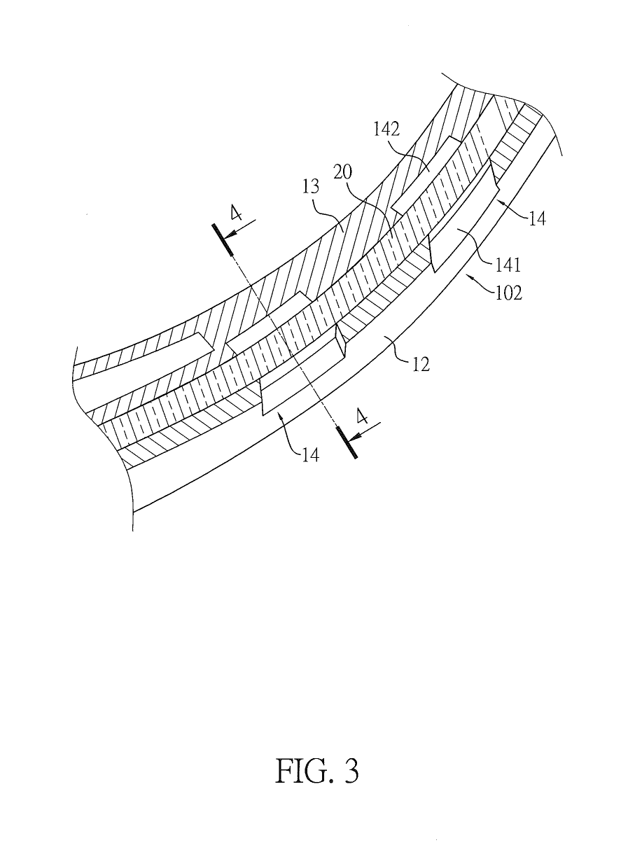

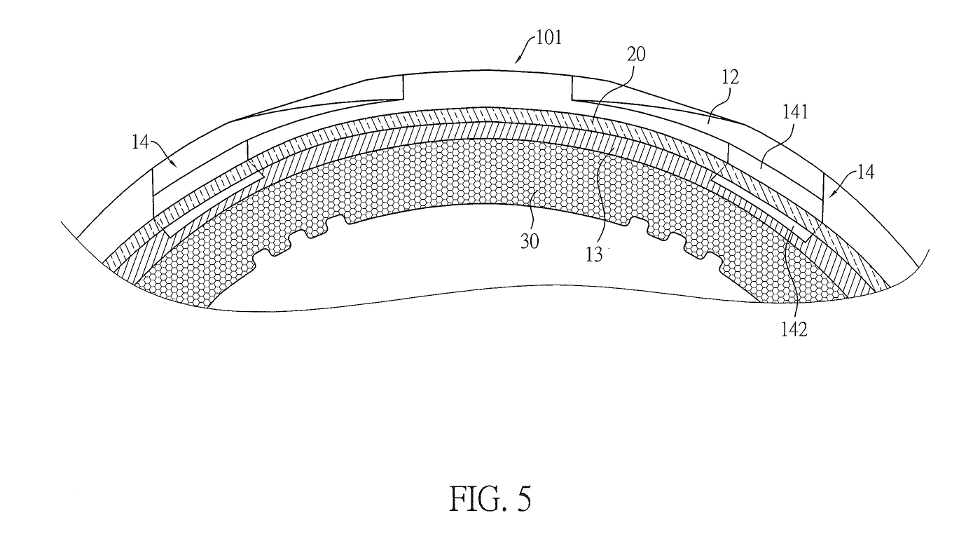

With further reference to FIGS. 3 and 5, the frame 10 has an upper frame portion 101, a lower frame portion 102, a rear peripheral surface, an inner peripheral surface, an inner peripheral recess 11, and multiple ventilation portions 14. The rear peripheral surface of the frame 10 faces toward a wearer of the goggles. The inner peripheral surface of the frame 10 surrounds a vision area AA of the goggles. The inner peripheral recess 11 is formed in and disposed along the inner peripheral surface of the frame 10, such that the frame 10 is divided into a front frame strip 12 and a rear frame strip 13.

The ventilation portions 14 are separately formed on the inner peripheral surface of the frame 10 and are disposed on the upper frame portion 101 and the lower frame portion 102 of the frame 10. Each of the ventilation portions 14 has a front ventilation hole 141 and a rear ventilation hole 142. The front ventilation hole 141 is formed through the front frame strip 12 of the frame 10. The rear ventilation hole 142 is formed through the rear frame strip 13 of the frame 10 and aligns with the front ventilation hole 141.

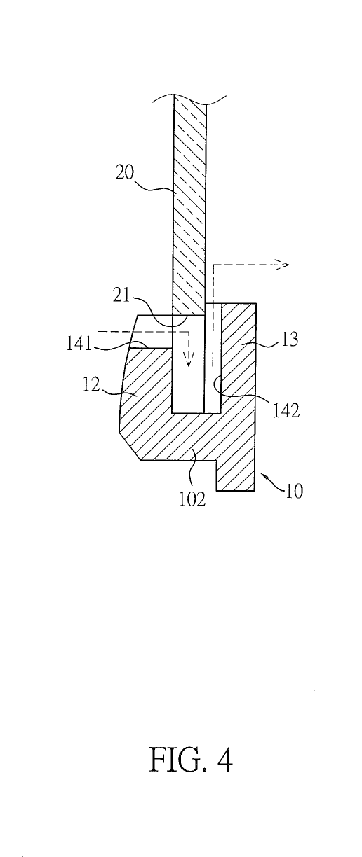

With further reference to FIG. 4, the lens 20 is mounted on the frame 10 and has a peripheral edge and multiple through holes 21. The peripheral edge of the lens 20 is inserted in the inner peripheral recess 11 of the frame 10, so as to mount the lens 20 on the frame 10. The through holes 21 are separately formed through the lens 20, are disposed along the peripheral edge of the lens 20, and respectively correspond in position to the ventilation portions 14 of the frame 10. Each of the through holes 21 communicates with the front ventilation hole 141 and the rear ventilation hole 142 of a corresponding one of the ventilation portions 14.

The cushion pad 30 is attached to the rear peripheral surface of the frame 10. When the wearer wears the goggles, the cushion pad 30 is fitted on a face of the wearer, so as to provide comfort to the wearer.

The strap 40 is elongated, is with an adjustable length, and has two ends respectively connected to two opposite sides of the frame 10. When the wearer wearers the goggles, the strap 40 pulls the frame 10, so the frame 10 stably abuts the face of the wearer. By adjusting the length of the strap 40, tightness of the strap 40 can be adjusted.

As shown in FIG. 4, with the ventilation portions 14 of the frame 10 and the through holes 21 of the lens 20, when the wearer wears the goggles, air outside an inner space defined between the lens 20 and the face of the wearer can flow into the space via the ventilation portions 14 that are disposed on the lower frame portion 102 of the frame 10 and the through holes 21 of the lens 20 that correspond in position to the ventilation portions 14 that are disposed on the lower frame portion 102. Then the air together with heat accumulated in the inner space flows out of the inner space via the ventilation portions 14 that are disposed on the upper frame portion 101 of the frame 10 and the through holes 21 of the lens 20 that correspond in position to the ventilation portions 14 that are disposed on the upper frame portion 101. Accordingly, the lens 20 can be prevented from fogging and vision of the wearer is not be blurred by fog.

The goggles as described have the following advantages. Since the ventilation portions 14 are formed on the inner peripheral surface of the frame 10 and the through holes 21 are disposed along the peripheral edge of the lens 20, formation of ventilation structure, i.e. the ventilation portions 14 and the through holes 21, does not require expanding a shape of the frame 10. Thus, the goggles have a compact volume and light weight and are comfortable for wearing.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. Goggles with a ventilation structure comprising:
a frame having
an inner peripheral surface;
an inner peripheral recess formed in and disposed along the inner peripheral surface of the frame to divide the frame into a front frame strip and a rear frame strip; and
multiple ventilation portions separately formed on the inner peripheral surface of the frame, and each of the ventilation portions having
a front ventilation hole formed through the front frame strip of the frame; and
a rear ventilation hole formed through the rear frame strip of the frame and aligning with the front ventilation hole;
a lens mounted on the frame and having
a peripheral edge inserted in the inner peripheral recess of the frame; and
multiple through holes separately formed through the lens, disposed along the peripheral edge of the lens, and respectively corresponding in position to the ventilation portions of the frame, and each of the through holes communicating with the front ventilation hole and the rear ventilation hole of a corresponding one of the ventilation portions; and
a strap being elongated and having two ends respectively connected to two opposite sides of the frame.

2. The goggles as claimed in claim 1, wherein
the frame has
an upper frame portion; and
a lower frame portion;
the ventilation portions of the frame are disposed on the upper frame portion and the lower frame portion of the frame.

3. The goggles as claimed in claim 1, wherein the goggles further comprise a cushion pad, and the cushion pad is attached to a rear peripheral surface of the frame.

4. The goggles as claimed in claim 2, wherein the goggles further comprise a cushion pad, and the cushion pad is attached to a rear peripheral surface of the frame.

5. The goggles as claimed in claim 1, wherein the strap is with an adjustable length.

6. The goggles as claimed in claim 2, wherein the strap is with an adjustable length.

7. The goggles as claimed in claim 3, wherein the strap is with an adjustable length.

8. The goggles as claimed in claim 4, wherein the strap is with an adjustable length.
